(19) United States
(12) Patent Application Publication
(10) Pub. No.: US 2003/0083260 A1
(43) Pub. Date: May 1, 2003

(54) Cryptococcal mannoproteins or equivalents thereof for use in modulating neutrophil migration

(76) Inventor: IIja Mohandas Hoepelman, Utrecht (NL)

Correspondence Address: TRASK BRITT, P.O. BOX 2550, SALT LAKE CITY, UT, 84110, US

(21) Appl. No.: 10/238,908

(22) Filed: Sep. 9, 2002

Related U.S. Application Data
(63) Continuation of application No. PCT/NL01/00192, filed on Mar. 8, 2001.

(30) Foreign Application Priority Data
Mar. 8, 2000 (EP) 00200826.6

(51) Int. Cl. A61K 38/16

(52) U.S. Cl. 514/12; 514/8

(57) ABSTRACT

The present invention is, among other things, concerned with suppressing neutrophil migration and the prevention of damage from neutrophil migration. A proteinaceous substance derivable from Cryptococcus neoformans and capable of interfering with neutrophil migration in a mammalian host. Also, functional equivalents of the proteinaceous substance and the use of this substance and/or equivalent in the preparation of a medicament or treatment of disese.

### CROSS-REFERENCE TO RELATED APPLICATION

This a continuation of pending PCT Internat'l Patent Appln. No. PCT/NL01/00192, filed Mar. 8, 2001, designating the United States of America, (published in English as WO 01/66574 A2 (Sep. 13, 2001), the contents of which are incorporated by this reference).

### TECHNICAL FIELD

The present invention relates to the field of diseases involving the immune system, in particular, diseases involving neutrophil migration.

### BACKGROUND

A number of serious illnesses involve neutrophil transmigration at one stage or the other. Such diseases include, among others, respiratory diseases as seen in Adult Respiratory Distress Syndrome (ARDS), acute traumas (especially trauma to the brain), infections where neutrophils invade (e.g., meningitis, peritonitis), reperfusion ischemia (stroke, heart attack, cardiopulmonary bypass surgery, spinal cord injury), skin diseases (contact dermatitis and psoriasis) and autoimmune diseases (e.g., rheumatoid arthritis and inflammatory bowel diseases such as ulcerative colitis and Crohn's disease). A very important disease for which no successful drug has yet been developed is bacterial meningitis.

Bacterial meningitis is a serious illness affecting annually in the US about 3 individuals per 100,000 (1). The case fatality isestimated at 25%, and a substantial percentage of the survivors will suffer permanent neurological impairment (2). Typically, a patient presents with serious clinical symptoms and a high leukocyte count in their cerebrospinal fluid (CSF; 3,4). Extensive literature evidence suggests that these—mainly neutrophil—leukocytes play an important adverse role in the pathogenesis of neurological damage (5-8).

It has been demonstrated that a high influx of neutrophil leukocytesinto the central nervous system (CNS) correlates with an unfavorableprognosis for neurological recovery (9, 10). Thus, several attempts havebeen made to reduce the inflammatory response (11). So far, the only studies published with adjuvant anti-inflammatory treatment in human bacterial meningitis, are clinical trials with corticosteroids added to the antibiotic regimen. Besides attenuation of TNF-levels, one of the proposed effects of corticosteroids involves the interference of neutrophil migration. Both for meningitis due to H. influenza type b as well as for pneumococcal meningitis this has become the standard of care. However, therapy with corticosteroids has many adverse effects.

As stated before, bacterial meningitis is not the only clinical situation where the influx of neutrophils has been associated with tissue damage (12-17). In serious brain injury (brain neurotrauma) and in ischemic events such as cerebrovascular ischemia, spinal cord injury/ischemia, myocardial infarction and intestinal ischemia, tissue reperfusion accompanied by neutrophil influx has repeatedly been correlated to tissue damage. Investigations into the role of neutrophils in non-infectious CNS damage have currently been intensified, since other therapeutical options in this field are very limited (18, 19).

Experimental animal models (20, 21) have been established to study various anti-inflammatory agents such as superoxide dismutase, transforming growth factor β, antibodies against adhesion molecules such as ICAM, caspase inhibitors, polysaccharides and cyclooxygenase inhibitors. Many of these strategies aim either directly at the reduction of the neutrophil influx or at (the release of) toxic neutrophil products into the CNS (22, 23). Even though the results look promising in animals, the effectiveness of these agents to reduce the migration of neutrophils into the CNS in the human situation remains to be established. Furthermore, there is considerable concern about potential side effects such as an adverse reaction to animal proteins (antibodies) and an increased susceptibility to infection through substantial interference with neutrophil function. Therefore, to our knowledge, the agents mentioned have not yet resulted in successful therapeutic tools that can be applied to the human situation. Thus, there is still an urgent need for new therapeutic means especially in CNS disease.

The present invention provides a novel means for suppressing neutrophil migration and thus a novel means for preventing damage resulting from neutrophil migration. The invention provides a proteinaceous substance derivable from Cryptococcus neoformans and capable of interfering with neutrophil migration in a mammalian host or a functional equivalent of such a proteinaceous substance for use as a pharmaceutical. Typically such proteinaceous substances in Cryptococcus itself are glycoproteins and more in particular mannoproteins. However, in other organisms functional equivalents (homologues) of these proteins may be found which have other characteristics but perform analogous functions as described herein. Also, based on the structure (3D) of the proteinaceous substances specifically disclosed herein, other molecules can be designed or produced that have at least one the same or similar function (in kind if not in amount). Also by mutating the specifically disclosed proteinaceous substances yet other functional equivalents may be prepared. These mutations are most easily created through the genetic information underlying the proteinaceous substances of the present invention, which are of course also part of the present invention. Preferred embodiments of the invention are mannoproteins 1, 2 and 4 of Cryptococcus or functional fragments or derivatives thereof. More preferred mannoprotein-4. In this respect functional fragments includes those fragments that have at least one similar or identical function of the original protein or carbohydrate moiety. Homologues are defined herein as proteins or carbohydrates having the same or similar function as the mannoproteins according to the invention but found in other organisms. Combinations of proteinaceous substances according to the invention may also be used. The afflictions for which the proteinaceous substances may be used are typically afflictions associated with an inflammatory response. Thus the invention includes the use of a proteinaceous substance or a functional equivalent thereof according to the invention in the preparation of a medicament for the treatment of diseases associated with inflammation, in particular for the treatment of a pathological condition or a disease involving neutrophil migration, more specifically a use whereby the inflammatory disease involves IL-8, fMLP, PAF or C5a regulation.

The invention further provides the use of a proteinaceous substance or a functional equivalent thereof according to the invention in the preparation of a medicament for the treatment of diseases associated with inflammation, in particular for the treatment of a pathological condition or a disease involving neutrophil migration, more specifically a use whereby the inflammatory disease involves TNF-α receptor or L-selectin down-regulation.

A preferred embodiment is a use according to the invention whereby the inflammatory disease is meningitis, in particular bacterial meningitis. Other uses include those whereby the pathological condition is serious brain injury, skin disease, allergy, ARDS, inflammatory bowel disease, rheumatoid arthritis (or another autoimmune disease), or an ischemic event such as myocardial infarct, cerebrovascular ischemia, or other cardiovascular diseases, intestinal ischemia, cardiovascular surgery, pulmonary ischemia or skeletal muscular ischemia.

The invention further provides a pharmaceutical composition comprising a proteinaceous substance or a functional equivalent thereof according to the invention together with a suitable means for administration. Typically these compositions may comprise further therapeutic agents such as an anti-inflammatory drug, a corticosteroid, or an antibiotic agent.

### DETAILED DESCRIPTION

In a particular preferred embodiment the invention provides the use of mannoproteins, more preferable mannoprotein-4 or a functional equivalent thereof in the preparation of a medicament for the treatment of a pathological condition or a disease involving neutrophil migration and in this sense as an anti-inflammatory agent. In inflammation, soluble factors called chemokines have been identified which attract various subtypes of leukocytes. The chemokine IL8 is a potent chemoattractant for neutrophils (24). As expected, patients with bacterial meningitis have high levels of IL8 as well as an increased number of neutrophils in their CSF. We recently described that patients who suffer from a fungal meningitis caused by Cryptococcus neoformans whose CSF typically contains very little leukocytes, had high levels of IL8 in their CSF (25). Therefore, in cryptococcal meningitis, an agent must be present that interferes with neutrophil migration toward IL8. Cryptococcal polysaccharides have been shown to interfere with neutrophil movement (26-29). We and others in literature initially focused on the fungal capsular polysaccharide glucuronoxylomannan (GXM) as the cryptococcal agent responsible for inhibition of neutrophil migration. Patients with cryptococcal infection have high titers of GXM in both serum and CSF during infection (30, 31). We demonstrated in vitro that GXM also interferes with neutrophil migration (chemotaxis) toward IL-8 if GXM is added directly to the leukocytes (25). We also demonstrated (32) that a low CSF leukocyte cell count in patients with cryptococcal meningitis shows a significant inverse correlation to a high GXM titer in serum (relative to the GXM titer in CSF). In a rabbit model for pneumococcal meningitis we have recently shown that intravenous administration of GXM not only delays entry of leukocytes (polymorphonuclear cells, PMN) into the CSF and brain, but more importantly reduces TNFα levels in the CSF and protects the brains of these rabbits against tissue damage in comparison to control animals (33). The exact mechanism how GXM interferes with neutrophil migration in cryptococcosis is not known. Interestingly, in the second part of 1999, we discovered that another class of cryptococcal proteins (34), the mannoproteins (MP) are even more powerful than GXM in reducing neutrophil migration, more in particular mannoprotein-4 (MP-4).

We compared GXM and three different MP's (MP-1, MP-2 and MP-4) for interference with different steps involved in transendothelial migration of PMN. 1., with the following results:

1.] GXM (35) and MP, more in particular MP-4, inhibit migration of PMN towards IL8, fMLP, C5a and PAF.

2.] GXM (36) and MP, more in particular MP-4, were shown to cause shedding of L-selectin (CD62L; the specific targeting of neutrophils to venules in inflamed tissue involves CD62L binding to E- and P-selectin on endothelial cells).

3.] GXM and MP, more in particular MP-4, caused up-regulation of CD11b and CD18 (the leukocyte function associated antigen-1, LFA-1).

4.] GXM and MP, more in particular MP-4, have intrinsic chemoattractive capacity.

5.] GXM and MP, more in particular MP-4, cause Ca-fluxes in leukocytes.

6.] MP, more in particular MP-4, causes down-regulation of TNFa receptors p55 and p75 on leukocytes.

7.] GXM and MP, more in particular MP-4, inhibit adhesion of neutrophils (PMN) to TNF stimulated endothelium.

8.] GXM and MP, more in particular MP-4, inhibit upregulation of adhesion molecules E-selectin (CD62E), VCAM, ICAM-1 on TNF stimulated endothelium.

9.] GXM and MP, more in particular MP-4, inhibit production of chemokines MCP-1, IL-8, MIP-1a and RANTES by TNF-stimulated endothelium.

In all assays MP turned out to be much more active than GXM; this holds true especially for MP-4 which could be used at 20- to 100-fold lower concentrations than GXM while still causing higher absolute stimulation levels (CD11b/18 upregulation, chemotaxis, Ca-flux) or repression levels (inhibition of chemotaxis levels, CD62L shedding).

Furthermore, we know that MP can be easily purified (37) and is not toxic; patients with cryptococcal meningitis can display relatively mild symptoms for a prolonged period of time despite the presence of MP, more in particular MP-4, in blood and CSF. Therefore MP is a potent therapeutic agent. Off course functional equivalents of MP will be capable of interfering with neutrophil migration. Since the protein and carbohydrate compositions of MP1, MP2 and MP4 (the most potent mannoprotein) closely resemble each other, they are placed here together as “MP”. Anti-inflammatory activity was detected in MP preparations, more in particular the MP-4 preparation. Thus MP, more in particular MP-4, and functional equivalents thereof can be applied in the treatment of diseases or pathological conditions involving neutrophil migration. Typically such diseases include infections and brain neurotrauma where neutrophils invade the tissue, diseases with damage due to reperfusion ischemia where neutrophils are attracted and cause secondary damage, intensive care patients with ARDS and other inflammatory diseases (rheumatoid arthritis and inflammatory bowel disease). MP, more in particular MP-4, will be especially useful in diseases involving IL8 regulation. Examples thereof are brain inflammatory disease such as meningitis—in particular bacterial meningitis—, neurotrauma (brain, spinal cord), lung disease (ARDS and COPD), cardiovascular disease (arteriosclerosis), allergic skin reactions (contact dermatitis, psoriasis), auto-immune diseases or the protection of the brain against toxic medication administered systemically (e.g. anti-cancer drugs). Other important applications will include the treatment of pathological conditions such as brain injury or ischemic events (ischemia—reperfusion after cardiopulmonary bypass surgery or other forms of cardiac surgery).

In a further embodiment the invention provides a pharmaceutical composition comprising MP or functional equivalents thereof together with a suitable means for administration. Typically such a composition will be given systematically. The dose of MP necessary to prevent neutrophil migration may vary, but will generally be between 250 mg/kg and 10 mg/kg, preferably between 1 mg/kg and 5 mg/kg. The composition may of course contain other drugs for the treatment of diseases involving neutrophil migration, such as anti-inflammatory drugs, corticosteroids or an antibiotic agent.

The invention will be explained in more detail in the following experimental part.

Isolation and Characterization of MP

Mannoproteins were purified from the supernatant of an a capsular mutant of C. neoformans, CAP 67 (E. S. Jacobson, Medical College of Virginia) to avoid contamination with GXM. Cryptococcal cultures were grown for 5 days in RPMI supplemented with gentamycin to prevent bacterial contamination. Supernatants were harvested by centrifugation and concentrated by ultrafiltration using a 3.5 kDa cut-off filter. Next, the retentate was applied to a concanavalin A column equilibrated in buffer A containing 10 mM Tris pH 7.2, 500 mM NaCl, 1 mM MgCl₂ and 1 mM CaCl₂. The column was washed with buffer A and stepwise eluted with a-methyl-D-mannose pyranoside in PBS. MP-1 and -2 eluted at 200 mM amDm, MP-4 eluted at 400 mM. MP-1 and MP-2 where further fractionated by anion exchange (DEAE) chromatography. Purified MP is finally recovered by lyophilization. Samples are analyzed by various methods to prove that none of the purified mannoproteins contains constituents other than those known to occur in MP (37): see table I.

Based on the purification schedule used to purify MP (as described above) we were able to draw the conclusion that MP, more in particular MP-4 purified by our methods was never present in the previously used MP preparations (29, 38), for the following reason. MP-4 purified by our method reveals a polydispersed signal migrating between 20 and 28 kDa on SDS-PAGE gels (37), whilst the culture filtrates (29) were concentrated using a 30 kDa cut-off cassette.

TABLE 1
Methylation analysis of mannoproteins obtained after DEAE
chromatography

| | Mole % of the residue | | |
|---|---|---|---|
| | present in the sample | | |
| Linkage Type | MP-1 | MP-2 | MP-4 |
| Terminal-Xyl | 4.3 | 3.0 | 1.2 |
| Terminal-Man | 24.7 | 31.3 | 32.6 |
| 2-O-Man | 18.6 | 31.7 | 43.0 |
| 4-O-Man | 7.3 | 1.7 | 1.6 |
| 6-O-Man | 5.5 | 8.5 | 8.2 |
| 6-O-Gal | 13.1 | 6.9 | 1.4 |
| 3-O-Man | 5.7 | 2.0 | 1.8 |
| 2,3-O-Man | 7.8 | 3.2 | 1.9 |
| 2,6-O-Man | 0.0 | 1.7 | 3.4 |
| 3,6-O-Gal | 8.3 | 4.5′ | 1.2 |
| 3,4-O-Gal | 2.8 | 2.4 | 0.0 |
| 3,6-O-Man | 0.0 | 1.2 | 2.6 |
| Terminal-Galf | 1.8 | 1.0 | 0.0 |
| Other | 0.0 | 1.0 | 0.7 |
| Total | 100 | 100 | 100 |

TABLE 2
Amino acid analysis of cryptococcal mannoproteins obtained after
DE 52 chromatography.

| | Mole % | | |
|---|---|---|---|
| Amino acid | MP-1 | MP-2 | MP-4 |
| Alanine | 18.7 | 13.21 | 14.75 |
| Arginine | 0.98 | 0.92 | 0.73 |
| Asparagine/Asp.acid | 8.37 | 11.37 | 10.93 |
| Glutamine/Glu.acid | 5.87 | 11.06 | 8.01 |
| Glycine | 4.67 | 3.84 | 4.74 |
| Isoleucine | 1.30 | 2.92 | 1.82 |
| Leucine | 1.96 | 4.61 | 3.28 |
| Methionine | 0.87 | 0.46 | 0.55 |
| Phenylalanine | 0.76 | 2.46 | 2.00 |
| Proline | 5.76 | 6.30 | 6.74 |
| Serine | 31.63 | 20.43 | 25.50 |
| Threonine | 15.98 | 17.20 | 16.76 |
| Tyrosine | 0.00 | 0.92 | 0.55 |
| Valine | 3.15 | 4.3 | 3.64 |

Effects of Cryptococcal GXM and MP-4 on Endothelial Cells

In all cases, confluent monolayers of human umbilical vein endothelial cells (HUVEC) were stimulated for 6 hours with 100 units/ml of TNFα after which the various assays were performed in the presence of polymyxin B.

I. GXM/MP-4 Inhibit Adhesion of Neutrophils (PMN) to TNF Stimulated Endothelium

In order to assess the effect of GXM/MP-4 on PMN adhesion, we compared adhesion of fluorescence labeled neutrophils—either GXM/MP-4 pretreated (1 hr at 37° C.; ng-mg/ml range) or untreated—to TNF-stimulated HUVEC in a static adhesion assay. After adhesion with PMN for 15 min. at 37° C. the monolayers were washed and the remaining adhesive PMN were fixed and counted in quadriple wells by means of fluorescence microscopy.

II GXM/MP4 Inhibit Upregulation of Adhesion Molecules E-selectin (CD62E), VCAM, ICAM-1 on TNF Stimulated Endothelium

In order to assess the effect of GXM/MP-4 on endothelial cells, we compared the expression of adhesion molecules on the surface of TNF-stimulated HUVEC cells—either GXM/MP-4 pretreated (6-24 hrs at 37° C.; ng-mg/ml range) or untreated—by measuring the levels of RNA encoding these molecules. For this, RNA isolated using the Trizol isolation method as described by the manufacturer was used as a template to produce cDNA. Specific cDNA encoding adhesion molecules was amplified by the polymerase chain reaction (PCR) technique using gene-specific PCR primers.

III GXM/MP-4 Inhibit Production of Chemokines MCP-1, IL-8, MIP-1α and RANTES by TNF-stimulated Endothelium

In order to assess the effect of GXM/MP-4 on endothelial cells, we compared the production of chemokines by TNF-stimulated HUVEC cells—either GXM/MP-4 pretreated (12-48 hrs at 37° C.; ng-mg/ml range) or untreated—by measuring the levels of chemokines present in the culture supernatant of endothelial cells cultured under these circumstances. GXM/MP-4 significantly inhibited the production of MCP-1, IL-8, MIP-1a and RANTES. Chemokine levels were determined using commercially available ELISA kits (R&D Systems).

### REFERENCES

1. Schlech W F I, Ward J I, Band J D et al. Bacterial meningitis in the US, 1978 through 1981. The national bacterial meningitis surveillance study. JAMA 1985; 253:1749-54.

2. Durand M L, Calderwood S B, Weber D J et al. Acute bacterial meningitis in adults: a review of 493 episodes. N.Engl.J.Med. 1993; 328:21-8.

3. Pfister H W, Fontana A, Tauber M G, Tomasz A and Scheld W M. Mechanisms of brain injury in bacterial meningitis: workshop summary

4. Spellerberg B and Tuomanen E I. The pathophysiology of pneumococcal meningitis. Ann.Med. 1994; 26:411-8.

5. Hartl R, Medary M B, Ruge M, Arfors K E and Ghajar J. Early white blood cell dynamics after traumatic brain injury: effects on the cerebral microcirculation. J.Cereb.Blood Flow Metab. 1997; 17:1210-20.

6. Hallenbeck J M. Cytokines, macrophages and leukocytes in brain ischemia. Neurology 1997; 49:S5-9.

7. Winquist R J and Kerr S. Cerebral ischemia-reperfusion injury and adhesion. Neurology 1997; 49:S23-6.

8. Yanada K, Camarata P J, Spellman S R et al. Antagonism of leukocyte adherence by synthetic fibronectin peptide V in a rat model of transient focal cerebral ischemia. Neurosurgery 1997; 40:557-63.

9. Tuomanen E I, Saukkonen K, Sande S, Cioffe C and Wright S D. Reduction of inflammation, tissue damage and mortality in bacterial meningitis in rabbits treated with monoclonal antibodies against adhesion-promoting receptors of leukocytes. J.Exp.Med. 1989; 170:959-69.

10. Saez Llorens X, Jafari H S, Severien et al. Enhanced attenuation of meningeal inflammation and brain edema by concomitant administration of anti-CD18 monoclonal antibodies and dexamethasone in experimental Haemophilus meningitis. J.Clin.Invest. 1991; 88:2003-11.

11. Quagliarello V J and Scheld W M. Treatment of bacterial meningitis. N.Engl.J.Med. 1997; 336:708-16.

12. Korthuis R J, Anderson D C and Granger D N. Role of neutrophil-endothelial cell adhesion in inflammatory disorders. J.Crit.Care 1994; 9: 47-71.

13. Tanaka M, Brooks S E, Richard V J et al. Effect of anti-CD18 antibody on myocardial neutrophil accumulation and infarct size after ischemia and reperfusion in dogs. Circulation 1993; 87:526-35.

14. Schoenberg M H, Poch B, Younes M et al. Involvement of neutrophils in postischemic damage to the small-intestine. Gut 1991; 32:905-12.

15. Kubes P, Hunter J and Granger D N. Ischemia/reperfusion-induced feline intestinal dysfunction: importance of granulocyte recruitment. Gastroenterology 1992; 103:807-12.

16. Sekido N, Mukaida N, Harada A et al. Prevention of lung reperfusion injury in rabbits by a monoclonal antibody against interleukin-8. Nature 1993; 365:654-7.

17. Petrasek P F, Liauw S, Romaschin A D and Walker P M. Salvage of postischemic skeletal muscle by monoclonal antibody blockade of neutrophil adhesion molecule CD18. J.Surg.Res. 1994; 56:5-12.

18. Clark W M and Zivin J A. Anti-leukocyte adhesion therapy: preclinical trials and combination therapy. Neurology 1997; 49:S32-8.

19. Tuomanen E. Adjunctive therapy of experimental meningitis: agents other than steroids. Antibiot.Chemother. 1992; 45:184-91.

20. Ley K, Cerrito M and Arfors K E. Sulfated polysaccharides inhibit leukocyte rolling in rabbit mesentery venules. Am.J.Physiol. 1991; 260:H1667-73.

21. Ley K, Linnemann G, Meinen M, Stoolman L and Gaehtgens P. Fucoidin, but not yeast polyphosphomannan PPME, inhibits leukocyte rolling in venules of the rat mesentery. Blood 1993; 81:177-85.

22. Adams D H and Lloyd A R. Chemokines: leukocyte recruitment and activation cytokines. Lancet 1997; 349:490-5.

23. Taub D D. Chemokine-leukocyte interactions: the voodoo that they do so well. Cytokine Growth Factor Reviews 1996; 7:355-76.

24. Bell M D, Taub D D and Perry V H. Overriding the brain's intrinsic resistance to leukocyte recruitment with intraparenchymal injections of recombinant chemokines. Neuroscience 1996; 74:283-92.

25. Chaka W S, Heyderman R, Gangaidzo I et al. Cytokine profiles in CSF of HIV-infected patients with cryptococcal meningitis: no leukocytosis despite high IL-8 levels. J.Infect.Dis. 1997; 176:1633-6.

26. Drouhet E and Segretain G. Inhibition de la migration leukocytaire in vitro par un polyoside capsulaire de Torulopsis (Cryptococcus) neoformans. Ann.Inst.Pasteur 1951; 81:674-6.

27. Dong Z M and Murphy J W. Effects of two varieties of Cryptococcus neoformans cells and culture filtrate antigens on neutrophil locomotion. Infect.Immun. 1995; 63:2632-44.

28. Dong Z M and Murphy J W. Intravascular cryptococcal culture filtrate (CneF) and its major component, glucuronoxylomannan, are potent inhibitors of leukocyte accumulation. Infect.Immun. 1995; 63:770-8.

29. Dong Z M and Murphy J W. Mobility of human neutrophils in response to Cryptococcus neoformans cells, culture filtrate antigen, and individual components of the antigen. Infect.Immun. 1993; 61:5067-77.

30. Mitchell T G and Perfect J R. Cryptococcosis in the era of AIDS—100 years after the discovery of Cryptococcus neoformans. Clinical Microbiology Reviews 1995; 8:515-48.

31. Eng R H K, Bishburg E and Smith S M. Cryptococcal infections in patients with acquired immune deficiency syndrome. Am.J.Med. 1986; 81:19-23.

32. Lipovsky M M, van Elden L J R, Walenkamp A M E, Dankert J and Hoepelman A I M. Does the capsular component glucuronoxylomannan of Cryptococcus neoformans impair transendothelial migration of leukocytes in patients with cryptococcal meningitis? J.Inf.Dis. 1998; 178:1231-32.

33. Lipovsky M M, Tsenova L, Coenjaerts F E, Kaplan G, Cherniak R and Hoepelman A I. Cryptococcal glucuronoxylomannan delays translocation of leukocytes across the blood-brain barrier in an animal model of acute bacterial meningitis. J.Neuroimmunol. 2000; 111: 10-14.

34. Cherniak R and Sundstrom J B. Polysaccharide antigens of the capsule of Cryptococcus neoformans. Infect.Immun. 1994; 62:1507-12.

35. Lipovsky M M, Gekker G, Hu S, Ehrlich L C, Hoepelman A I M and Peterson P K. Cryptococcal glucuronoxylomannan induces interleukin 8 (IL8) production by human microglia but inhibits neutrophil migration towards IL8. J.Infect.Dis. 1998; 177:260-3.

36. Dong Z M and Murphy J W. Cryptococcal polysaccharides induce L-selectin shedding and tumor necrosis factor receptor loss from the surface of human neutrophils. J.Clin.Invest. 1996; 97:689-98.

37. Walenkamp A M E, Chaka W S, Verheul A F M, Vaishnav V V, Cherniak R, Coenjaerts F E J and Hoepelman I M. Cryptococcus neoformans and its cell wall components induce similar cytokine profiles in human peripheral blood mononuclear cells despite differences in structure. FEMS Imm and Med. Microbiology 1999; 26:309-18

38. Orendi J M, Verheul A F M, de Vos N M, Visser M R, Snippe H, Cherniak R, Vaishnav V V, Rijkers G T and Verhoef J. Mannoproteins of Cryptococcus neoformans induce proliferative response in human peripheral blood mononuclear cells and enhance HIV-1 replication. Clin.Exp.Immunol. 1997;107:293-9.

What is claimed is:

1. A pharmaceutical composition comprising:
a proteinaceous substance derivable from Cryptococcus neoformans and capable of interfering with neutrophil migration in a mammalian host or a functional equivalent of said proteinaceous substance; and
a suitable means for administration.

2. The pharmaceutical composition of claim 1 wherein the proteinaceous substance is a glycoprotein.

3. The pharmaceutical composition of claim 1 or 2, wherein the proteinaceous substance is a mannoprotein or a functional equivalent thereof.

4. The pharmaceutical composition of claim 3, wherein the proteinaceous substance is selected from the group consisting of mannoprotein 4, a functional fragment of mannoprotein 4, and a functional homologue of mannoprotein 4.

5. The pharmaceutical composition of claim 3 wherein the proteinaceous substance is selected from the group consisting of mannoprotein 1, mannoprotein 2, mannoprotein 4, a functional fragment of mannoprotein 1, mannoprotein 2, or mannoprotein 4, a functional homologue of mannoprotein 1, mannoprotein 2, or mannoprotein 4, and a combination of any thereof.

6. The pharmaceutical composition of claim 1, claim 2, claim 3, claim 4, or claim 5 further comprising an anti-inflammatory drug.

7. The pharmaceutical composition of claim 1, claim 2, claim 3, claim 4, claim 5, or claim 6, further comprising a corticosteroid.

8. The pharmaceutical composition of claim 1, claim 2, claim 3, claim 4, claim 5, claim 6, or claim 7, further comprising an antibiotic agent.

9. A method of treating a disease associated with inflammation in a subject, said method comprising:
administering to the subject a proteinaceous substance derivable from Cryptococcus neoformans and capable of interfering with neutrophil migration in a mammalian host or a functional equivalent of said proteinaceous substance.

10. The method according to claim 9 wherein the proteinaceous substance is a glycoprotein.

11. The method according to claim 9 or 10, wherein the proteinaceous substance is a mannoprotein or a functional equivalent thereof.

12. The method according to claim 11 wherein the proteinaceous substance is selected from the group consisting of mannoprotein 1, mannoprotein 2, mannoprotein 4, a functional fragment of mannoprotein 1, mannoprotein 2, or mannoprotein 4, a functional homologue of mannoprotein 1, mannoprotein 2, or mannoprotein 4, and a combination of any thereof.

13. A method of treating a subject suffering from a pathological condition or a disease involving neutrophil migration, the method comprising:
administering to the subject a proteinaceous substance derivable from Cryptococcus neoformans and capable of interfering with neutrophil migration in a mammalian host or a functional equivalent of said proteinaceous substance.

14. The method according to claim 13 wherein the proteinaceous substance is a glycoprotein.

15. The method according to claim 13 or 14, wherein the proteinaceous substance is a mannoprotein or a functional equivalent thereof.

16. The method according to claim 15 wherein the proteinaceous substance is selected from the group consisting of mannoprotein 1, mannoprotein 2, mannoprotein 4, a functional fragment of mannoprotein 1, mannoprotein 2, or mannoprotein 4, a functional homologue of mannoprotein 1, mannoprotein 2, or mannoprotein 4, and a combination of any thereof.

17. The method according to claim 9 or 13, wherein the disease is an infection.

18. The method according to claim 9 wherein the inflammatory disease involves IL8,fMLP, PAF or C5a regulation or TNF-α receptor and/or L-selectin down-regulation.

19. The method according to any one of claims 9 through 18, wherein the inflammatory disease is meningitis.

20. The method according to claim 13 wherein the pathological condition is selected from the group consisting of serious brain injury, skin disease, allergy, ARDS, inflammatory bowel disease, rheumatoid arthritis, or an ischemic event.

21. The method according to claim 20 wherein the pathological condition is an ischemic event selected from the group consisting of a myocardial infarct, cerebrovascular ischemia, other cardiovascular diseases, intestinal ischemia, cardiovascular surgery, pulmonary ischemia, and skeletal muscular ischemia.
